(19) United States
(12) Patent Application Publication
(10) Pub. No.: US 2008/0057140 A1
(43) Pub. Date: Mar. 6, 2008

(54) Use of a Lignan for the Manufacture of a Composition for Preventing or Alleviating of Symptoms Relating to Estrogen Deficiency

(76) Inventor: Mikko Unkila, Piikkio (FI)

Correspondence Address: JAMES C. LYDON, 100 DAINGERFIELD ROAD, SUITE 100, ALEXANDRIA, VA, 22314, US

(21) Appl. No.: 11/813,589

(22) PCT Filed: Nov. 17, 2005
(86) PCT No.: PCT/FI05/00490

§ 371 Date: Sep. 25, 2007

Related U.S. Application Data
(60) Provisional application No. 60/642,148, filed on Jan. 10, 2005.

(51) Int. Cl. A61K 36/00; A61K 31/047; A61K 31/341; A61P 5/24; A61K 31/343

(52) U.S. Cl. 424/725; 514/456; 514/468; 514/473; 514/732

(57) ABSTRACT

The use of a lignan, which is a plant lignan, a metabolite thereof or a combination of both, for the manufacture of a composition for preventing or alleviating of symptoms relating to estrogen deficiency in an individual. Also disclosed is the use of a plant lignan capable of being a precursor for enterolactone or another metabolite of the plant lignan, for the manufacture of a composition useful for increasing the level of enterolactone or another metabolite of a plant lignan in an individual's serum, where the individual suffers from or is at risk of estrogen deficiency.

### FIELD OF THE INVENTION

This invention relates to a method for preventing or alleviating of symptoms related to estrogen deficiency in individuals, especially menopausal or postmenopausal symptoms associated with, but not limited to age-related decrease in estrogen hormone production in women. Particularly, the invention relates to prevention or alleviating of climacteric symptoms such as hot flushes in women during and after the menopause.

### BACKGROUND OF THE INVENTION

The publications and other materials used herein to illuminate the background of the invention, and in particular, cases to provide additional details respecting the practice, are incorporated by reference.

Age, diseases, surgical operations, drug treatments and environmental factors amongst the other can interfere with the physiological hormonal balance (hormone synthesis and degradation, interference with hormonal signalling) leading to aberrant, often diminished hormonal activity in the body. In connection to this, in menopausal women a decrease in estrogen hormone production often causes insomnia, mood swings, forgetfulness and hot flashes. These symptoms are directly linked to a decline and/or erratic production of estrogen by the ovaries. Nearly all women find the menopause-associated vasomotor (hot flashes) and other symptoms a phenomenon which considerably decreases quality of life. Doctors often recommend hormone replacement therapy (HRT) for relief of these symptoms; however, a recent study by the Women's Health Initiative (WHI) disclosed adverse effects associated with HRT. The data demonstrated that long-term HRT increased the risk of breast cancer, stroke, pulmonary embolism and coronary heart disease. Thus, there is a clear need for alternative treatments to ameliorate the signs and symptoms associated with the decline of estrogen production/action in women.

Driven by the realization that HRT is not as safe and/or effective as previously thought, there is a growing interest in the in the plant-derived estrogens, called phytoestrogens to provide an alternative to the HRT.

Lignans are considered as phytoestrogens, and they are defined as a class of phenolic compounds possessing a 2,3-dibenzylbutane skeleton. They are formed by coupling of monomeric units called precursors such as cinnamic acid, caffeic, ferulic, coumaric, and gallic acids (1). Lignans are widely distributed in plants. They can be found in different parts (roots, leafs, stem, seeds, fruits) but mainly in small amounts. In many sources (seeds, fruits) lignans are found as glycosidic conjugates associated with fiber component of plants. The most common dietary sources of mammalian lignan precursors are unrefined grain products. The highest concentrations in edible plants have been found in flaxseed, followed by unrefined grain products, particularly rye.

Considerable amounts of lignans are also found in coniferous trees. The type of lignans differs in different species and the amounts of lignans vary in different parts of the trees. The typical lignans in heart wood of spruce (Picea abies) are 7-hydroxymatairesinol (HMR), a-conidendrin, conidendrinic acid, matairesinol, isolariciresinol, secoisolariciresinol, liovile, picearesinol, lariciresinol and pinoresinol (2). The far most abundant single component of lignans in spruce is 7-HMR, about 60 percent of total lignans, which occurs mainly in unconjugated free form.

Plant lignans such as 7-hydroxymatairesinol, matairesinol and secoisolariciresinol, are converted by gut microflora to mammalian lignans, enterolactone or enterodiol (3; WO 00/59946). A recent study (4) shows also that matairesinol, secoisolariciresinol, lariciresinol and pinoresinol glucoside were to be converted to enterolactone.

Enterolactone is known to possess many valuable therapeutical properties. Urinary excretion and serum concentrations of enterolactone are low in women diagnosed with breast cancer (5, 6) and have lower mineral density of the bone (7) suggesting that this lignan is chemopreventive. The direct binding of enterolactone to the estrogen receptor alpha (FIG. 1) or the inhibition of steroid metabolizing aromatase by enterolactone would suggest a mechanism by which consumption of lignan-rich plant food might contribute to reduction of estrogen-dependent diseases, such as breast cancer (8, 9).

A recent study (10) disclosed that diet supplemented with flaxseed was effective in ameliorating the climacteric symptoms in menopausal women. Flax has a high concentration of secoisolariciresinol, which is readily converted into mildly estrogenic enterolactone in humans. Diets fortified with flax also readily elevate circulating enterolactone levels in humans (11, 12 and references therein). Collectively, these finding suggest that lignans that can serve as enterolactone precursors may be effective in ameliorating menopausal symptoms and osteoporosis.

Methods for the synthesis of enterolactone has been disclosed in the literature (13). However, isolated mammalian lignans such as enterolactone, have not so far been available in sufficient amounts to be used in animal experiments or clinical trials. The only possibility to increase mammalian lignan supply has been to increase the consumption of fiber-rich food items such as flaxseed.

The international patent publication WO 00/59946 discloses that 7-hydroxymatairesinol is efficiently converted to enterolactone in vivo and thus useful to increase the level of enterolcatone. The publication also indicates that 7-hydroxymatairesinol can be effective as such due to its antioxidative activity in vitro. This publication discloses usefulness of hydroxymatairesinol in the prevention of cancers such as breast cancer, prostate cancer and colon cancer, non-cancer, hormonal dependent diseases such as lower urinary tract symptoms, urethral dyssynergia, bladder instability, bladder outlet obstruction, benign prostatic hyperplasia, and gynecomastia in men, and cardiovascular diseases resulting from oxidized LDL in serum.

### SUMMARY OF THE INVENTION

According to one aspect, this invention concerns a method for preventing or alleviating of symptoms relating to estrogen deficiency in an individual, said method comprising administering to the individual an effective amount of a lignan which is a plant lignan, a metabolite thereof or a combination of both.

According to another aspect, the invention concerns a method for increasing the level of enterolactone or another metabolite of a plant lignan in an individual's serum wherein said individual suffers from or is at risk of estrogen deficiency, said method comprising administering to the individual an effective amount of a plant lignan capable of being a precursor for enterolactone or another metabolite of said plant lignan.

### BRIEF DESCRIPTION OF THE FIGURES

FIG. 1 shows competition of different phytoestrogens in the recombinant estrogen receptor alpha binding test.

FIG. 2 shows plasma concentrations (mean +SD) of enterolactone in human subjects treated with 315 mg/day (divided in three 105 mg portions) of 7-hydroxymatairesinol for 29 days. The data for experimental days 1 and 29 is shown. On the x-axis, the time (hrs) after the first 105 mg dose is shown. The arrows denote the administration of the three 105 mg HMRlignan™ (hydroxymatairesinol) doses during the test days 1 and 29. N=6.

### DETAILED DESCRIPTION OF THE INVENTION

The menopausal decrease of ovarian function and estrogen production is often compensated by hormonal replacement with estrogen and progestin. However, because of growing awareness of potential side effects of the HRT, the women at menopause refuse to take this medication. This often leads to more severe vasomotor (climacteric symptoms) and risk of bone fractures due to accelerated loss of bone mineral content after the menopause.

Since several plant derived compounds called phytoestrogens can mimic the action of estrogen in at least pharmacological test systems, there is a growing interest and utilization of these products in eg. menopausal and postmenopausal women.

The symptoms and diseases which can be prevented by the method according to this invention are, for example, menopausal vasomotor symptoms, mood swings, insomnia and dryness of the mucosa of lower urinary tract.

Furthermore, long-term treatment with lignans may also inhibit the development of osteoporosis by delaying the loss of bone minerals because of loss of estrogen function.

As a first alternative, a decreased level of mammalian lignans, especially enterolactone, in blood appears as a prerequisite for a risk to develop more severe symptoms and conditions during the menopause. Therefore, promoting the diet of meno- or postmenopausal women with a plant lignan and thereby elevating blood enterolactone concentration may be effective in alleviating such symptoms. By elevating the blood enterolactone concentration to suitable level (e.g. 30-200 nMol/l), can have a pronounced alleviating effect on the menopausal symptoms/conditions.

As a second alternative, lignans have several putative beneficial properties as nutritional supplements (e.g. they are antioxidants), thus meno/postmenopausal women may also benefit direct effects associated with plant lignans. It may also be true, that addition of a plant lignan (eg. 7-hydroxymatairesinol) in daily diet may elicit beneficial health effects without conversion to mammalian lignans such as enterolactone. This assumption should be taken into account since there is no final proof that plant lignans have to be converted into mammalian lignan (enterolactone) before their curative effects towards menopausal/postmenpausal symptoms. Further, estrogen is considered as a balancing/modulatory mediator for the immune system and loss of estrogen production during menopause is also associated with increased risk of developing certain autoimmune and other immunological disease (cardiovascular diseases, systemic lupus erythematosus, Alzheimer's disease). Thus, by their strong anti-oxidant and anti-inflammatory actions, women at or after menopause can have advantage from this property of lignans.

As a third alternative, the mammalian lignan, e.g. enterolactone or enterodiol, can be administered as such to the individual.

As fourth alternative, plant and/or mammalian lignan can be combined with compounds from other classes of phytoestrogens such as isoflavones (eg. genistein from soy), coumestans, red clover, which are also known for their putative beneficial effects in alleviating menopausal symptoms. It may be anticipated that the therapeutic effects of such combinations to be greater than from individual components administered alone.

Preferred plant lignans are, for example, 7-hydroxymatairesinol, allohydroxymatairesinol, matairesinol, lariciresinol, secoisolariciresinol, isolariciresinol, oxomatairesinol, conidendrin, conidendric acid, pinoresinol, pinoresinol glucoside, liovil, picearesinol, nortrachelogenin, arctigenin, and their geometric isomers and stereoisomers, salts and adducts, and mixtures.

Particularly preferred are the plant lignans hydroxymatairesinol, matairesinol, lariciresinol, secoisolariciresinol, pinoresinol and pinoresinol glucoside, and their geometric isomers and stereoisomers, salts and adducts, and mixtures. These lignans have been shown a good ability to be converted into enterolactone.

Preferred mammalian lignans are enterolactone and enterodiol, especially enterolactone.

The lignan to be administered shall in this text be understood to cover any geometric isomer or stereoisomer or any mixture of isomers, such as racemates, of these compounds. Salts, adducts and complexes of the compounds shall also be understood to be covered by the term.

The lignans to be used in this invention can be supplied in the form of a pharmaceutical preparation, dietary supplement, clinical nutrition formula or as a functional food. According to a particularly preferred embodiment, the lignan is administered as a dietary supplement for clinical nutritional purposes to the coeliac patients.

The pharmaceutical preparation according to this invention is preferably an oral formulation. The required amount of the active compound or mixture of compounds will vary with the compound and the particular condition to be prevented. A typical dose ranges from about 10 to about 2000 mg per day and adult person, preferably 100 to 600 mg per day and adult person. Typical dosage forms include, but are not limited to, oral dosage forms such as powders, granules, capsules, tablets, caplets, lozenges, liquids, elixirs, emulsions and suspensions. All such dosage forms may include conventional carriers, diluents, excipients, binders and additives known to those skilled in the medicinal and pharmaceutical arts.

The pharmaceutical or other formula carriers typically employed may be solid or liquid. Thus, for example, solid carriers include polysaccarides such as lactose, sucrose, gelatin, agar, while liquid carriers include aqueous solutions of salts, polysaccarides, complexing agents, surfactants, syrups, vegetable oils such as peanut oil or olive oil, and certain alcohols. However, any acceptable solid or liquid carrier can be used in the pharmaceutical preparation or other dietary or nutrition formula to be administered according to this invention.

A typical food product, suitable for use in the methods according to this invention, is especially a functional food, a nutritional supplement, a nutrient, a pharmafood, a nutraceutical, a health food, a designer food or any food product. A suitable concentration of the active compound the food product is, for example, 5 to 1000 mg of active compound per 100 g of food product, preferably about 10 to 100 mg of active compound per 100 g of food product.

### EXPERIMENTAL

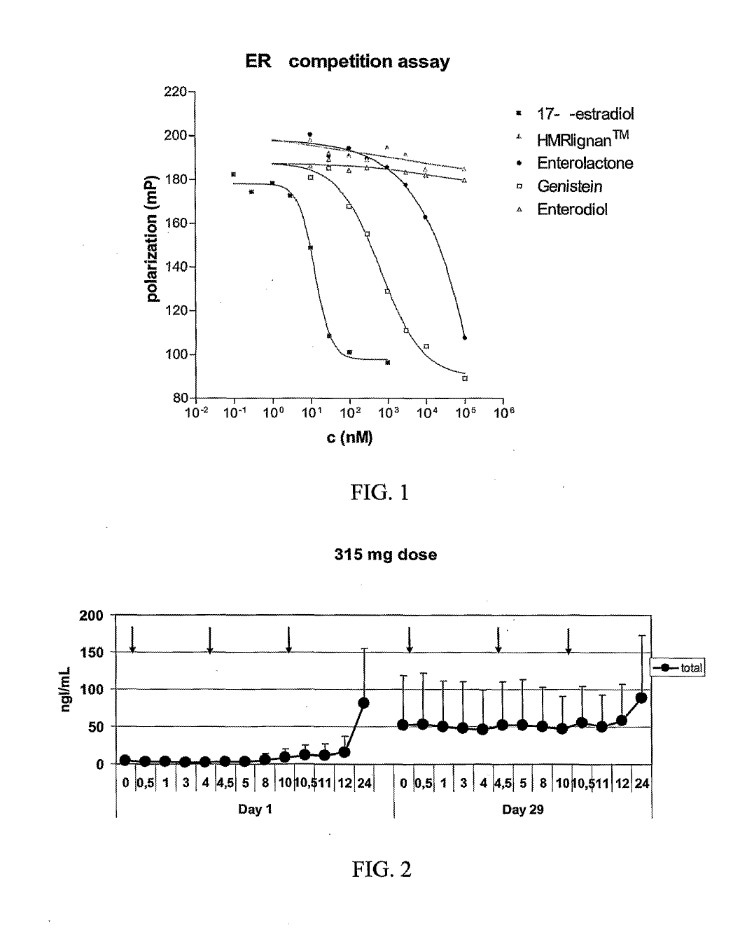

The efficiency of enterolactone as phytoestrogen was tested in the estrogen receptor binding assay with recombinant estrogen receptor alpha. The results are shown in FIG. 1. Briefly, the test is performed with a commercially available estrogen receptor alpha binding kit (Invitrogen/Pan Vera Corp., Estrogen receptor competitor assay green # P2614, P2698). The test compounds and control compound dissolved in DMSO at 10⁻²M stock solution. The test compounds and reagents are added to a 96 well microtiterplate plate for preparing of serial dilutions and testing the binding activity. The results are obtained by measuring changes in fluorescence polarization induced by test compounds, reflecting compounds ability to bind estrogen receptor alpha. The compounds are tested at 1, 10, 100, 1000 and 10 000 nM concentration and results are obtained with an Tecan Ultra Evolution microplate reader (Switzerland). An estrogen receptor alpha displacement curve is obtained (Prism software, GraphPad Inc.), and an IC50 concentration can be extrapolated from the curve.

The efficiency of 7-hydroxymatairesinol as enterolactone precursor was also recently confirmed in human subjects and results showed that 4 week treatment with 7-hydroxymatairesinol in capsule form resulted in sustained increase in circulating enterolactone concentration. The results are shown in FIG. 2. In brief, healthy male volunteers ingested capsule preparation of 7-hydroxymatairesinol, at total daily dose of 315 mg, divided in three 105 mg portion for morning, afternoon and evening dose. The subjects ingested the product continuously for 29 days. The level of enterolactone was measured from plasma at 0, 0.5, 1, 3, 4, 4.5, 5, 8, 10, 10.5, 12 and 24 hours after the first daily 105 mg 7-HMR dose on experimental test days 1 and 29. The levels were measured with a high-performance liquid chromatography coupled with tandem mass spectrometer system (Applied Biosystems Inc.).

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

### REFERENCES

1. Ayres D, and Loike, J. Lignans: Chemical, bilogical and clinical properties. Cambridge university press, 1990.
2. Ekman R: Distribution of lignans in Norway spruce. Acta Academiae Aboensis, Ser B, 39:1-6, 1979.
3. Axelson M, Sjövall J, Gustafsson B E and Setchell K D R: Origin of lignans in mammals and identification of a precursor from plants. Nature, 298: 659-660, 1982.
4. Heinonen S, Nurmi T, Liukkonen K, Poutanen K, Wahala K, Deyama T, Nishibe S. Adlercreutz H (2001) In vitro metabolism of plant lignans: new precursors of mammalian lignans enterolactone and enterodiol. J Agric Food Chem, 49, 3178-86.
5. Ingram D, Sanders K. Kolybaba M and Lopez D. Case-control study of phyto-oestrogens and breast cancer. Lancet, October 4; 350(9083): 990-994, 1997.
6. Hultén K, Adlercreutz H, Winkvist A, Lenner P, Hallmans G and \dot{A}gren \dot{A}. Low levels of phyto-estrogens in blood as risk factor for breast cancer. In: COST 916 Workshop ‘Phyto-oestrogens: exposure, bioavailability, health benefits and safety concerns’, 1998
7. Kim M K, Chung B C, Yu V Y, Nam J H, Lee H C, Huh K B, Lim S K. (2002) Relationships of urinary phyto-oestrogen excretion to BMD in postmenopausal women. Clin Endocrinol 56:321-8.
8. Adlercreutz H, Bannwart C, Wähälä K, Mäkelä T, Brunow G, Hase T, Arosemena P J, Kellis J T, and Vickery L E: Inhibition of human aromatase by mammalian lignans and isoflavonoid phytoestrogens. J Steroid Biochem Mol Biol, 44: 147-153, 1993.
9. Wang C, Mäkelä T, Hase T, Adlercreutz H and Kurzer M S: Lignans and flavonoids inhibit aromatase enzyme in human adipocytes. J Steroid Biochem Molec Biol, 50: 205-212, 1994.
10. Lemay A, Dodin S, Kadri N, Jacques H, Forest J C. (2002) Flaxseed dietary supplement versus hormone replacement therapy in hypercholesterolemic menopausal women. Obstet Gynecol. 100:495-504.
11. Tarpila S, Aro A, Salminen I, Tarpila A, Kleemola P, Akkila J, Adlercreutz (20029 The effect of flaxseed supplementation in processed foods on serum fatty acids and enterolactone. Eur J Clin Nutr. 56:157-65.
12. Nesbitt P D, Lam Y, Thompson L U. (1999). Human metabolism of mammalian lignan precursors in raw and processed flaxseed. Am J Clin Nutr. 69:549-55.
13. M B Groen and J Leemhuis, Tetrahedron Letters 21, 5043 (1980) and G Cooley et al., ibid 22, 349 (1981).

1. The use of a lignan which is a plant lignan, a metabolite thereof or a combination of both for the manufacture of a composition for preventing or alleviating of symptoms relating to estrogen deficiency in an individual.

2. The use according to claim 1 wherein the symptoms are menopausal or postmenopausal symptoms.

3. The use according to claim 2 wherein the symptoms are climacteric symptoms.

4. The use according to claim 3 wherein the symptoms are hot flushes.

5. The use according to claim 2 wherein the symptoms are vaginal dryness, vaginal atrophy, atrophy of the lower urinary tract, loss of bone mineral content, menopausal vasomotor symptoms, mood swings, insomnia, osteoporosis or any other menopause associated condition.

6. The use according to claim 1 wherein the estrogen deficiency is caused by medication or a surgical operation.

7. The use according to claim 1, wherein the plant lignan is 7-hydroxymatairesinol, allohydroxymatairesinol, matairesinol, lariciresinol, secoisolariciresinol, isolariciresinol, oxomatairesinol, conidendrin, conidendric acid, pinoresinol, pinoresinol glucoside, liovil, picearesinol, nortrachelogenin, arctigenin, a geometric isomer or a stereoisomer, salt or adduct thereof or a mixture thereof.

8. The use according to claim 7 wherein the plant lignan is wood-derived 7-hydroxymatairesinol, a geometric isomer, stereoisomer, salt or adduct thereof.

9. The use according to claim 1, wherein metabolite is enterolactone or/and enterodiol.

10. The use of a plant lignan capable of being a precursor for enterolactone or another metabolite of said plant lignan, for the manufacture of a composition useful for increasing the level of enterolactone or another metabolite of a plant lignan in an individual's serum, wherein said individual suffers from or is at risk of estrogen deficiency.

11. The use according to claim 10 wherein the plant lignan is 7-hydroxymatairesinol, allohydroxymatairesinol, matairesinol, lariciresinol, secoisolariciresinol, isolariciresinol, oxomatairesinol, conidendrin, conidendric acid, pinoresinol, pinoresinol glucoside, liovil, picearesinol, nortrachelogenin, arctigenin, a geometric isomer or a stereoisomer, salt or adduct thereof or a mixture thereof.

12. The use according to claim 11 wherein the plant lignan is wood-derived hydroxymatairesinol, a geometric isomer, a stereoisomer, a salt or adduct thereof.

13. The use according to claim 1, wherein also another phytoestrogen, particularly an isoflavone and/or red clover is administered.

14. A method for preventing or alleviating of symptoms relating to estrogen deficiency in an individual, said method comprising administering to the individual an effective amount of a lignan which is a plant lignan, a metabolite thereof or a combination of both.

15. The method according to claim 14 wherein the symptoms are menopausal or postmenopausal symptoms.

16. The method according to claim 15 wherein the symptoms are climacteric symptoms.

17. The method according to claim 16 wherein the symptoms are hot flushes.

18. The method according to claim 15 wherein the symptoms are vaginal dryness, vaginal atrophy, atrophy of the lower urinary tract, loss of bone mineral content, menopausal vasomotor symptoms, mood swings, insomnia, osteoporosis or any other menopause associated condition.

19. The method according to claim 14 wherein the estrogen deficiency is caused by medication or a surgical operation.

20. The method according to claim 14, wherein the plant lignan is 7-hydroxymatairesinol, allohydroxymatairesinol, matairesinol, lariciresinol, secoisolariciresinol, isolariciresinol, oxomatairesinol, conidendrin, conidendric acid, pinoresinol, pinoresinol glucoside, liovil, picearesinol, nortrachelogenin, arctigenin, a geometric isomer or a stereoisomer, salt or adduct thereof or a mixture thereof.

21. The method according to claim 20 wherein the plant lignan is wood-derived 7-hydroxymatairesinol, a geometric isomer, stereoisomer, salt or adduct thereof.

22. The method according to claim 14, wherein metabolite is enterolactone or/and enterodiol.

23. A method for increasing the level of enterolactone or another metabolite of a plant lignan in an individual's serum wherein said individual suffers from or is at risk of estrogen deficiency, said method comprising administering to the individual an effective amount of a plant lignan capable of being a precursor for enterolactone or another metabolite of said plant lignan.

24. The method according to claim 23 wherein the plant lignan is 7-hydroxymatairesinol, allohydroxymatairesinol, matairesinol, lariciresinol, secoisolariciresinol, isolariciresinol, oxomatairesinol, conidendrin, conidendric acid, pinoresinol, pinoresinol glucoside, liovil, picearesinol, nortrachelogenin, arctigenin, a geometric isomer or a stereoisomer, salt or adduct thereof or a mixture thereof.

25. The method according to claim 24 wherein the plant lignan is wood-derived hydroxymatairesinol, a geometric isomer, a stereoisomer, a salt or adduct thereof.

26. The method according to claim 14, wherein also another phytoestrogen, particularly an isoflavone and/or red clover is administered.
